(19) United States
(12) Patent Application Publication
(10) Pub. No.: US 2010/0317663 A1
(43) Pub. Date: Dec. 16, 2010

(54) ANILINOPYRIDINES AS INHIBITORS OF FAK

(76) Inventors: Jerry Leroy Adams, Collegeville, PA (US); Thomas H. Faitg, Collegeville, PA (US); Neil W. Johnson, Collegeville, PA (US); Xin Peng, Collegeville, PA (US)

Correspondence Address: GlaxoSmithKline;GLOBAL PATENTS -US, UW2220, P. O. BOX 1539, KING OF PRUSSIA, PA, 19406-0939, US

(21) Appl. No.: 12/867,794

(22) PCT Filed: Feb. 19, 2009
(86) PCT No.: PCT/US09/34443

§ 371 Date: Aug. 16, 2010

Related U.S. Application Data
(62) Division of application No. 61/029,609, filed on Feb. 19, 2008.

(51) Int. Cl. A61K 31/5377; C07D 401/12; C07D 413/12; A61K 31/4439; A61P 35/00

(52) U.S. Cl. 514/235.5; 546/277.7; 544/131; 514/339

(57) ABSTRACT

The present invention relates to a compound of formula (I):

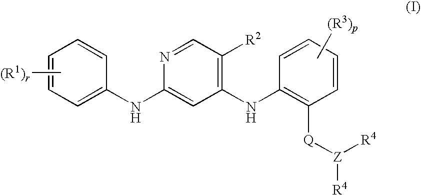

or a pharmaceutically acceptable salt thereof, wherein R¹-R⁴, Q, Z, r, and p are as defined herein. Compounds of the present invention are useful in the treatment of diseases associated with FAK overexpression, including proliferative diseases.

This invention relates to a class of anilinopyridines that inhibit Focal Adhesion Kinase (FAK), as well as compositions thereof. Compounds of the present invention are useful in the treatment of proliferative diseases.

Tyrosine kinases play an important role in the regulation of many cell processes including cell proliferation, cell survival, and cell migration. It is known that certain tyrosine kinases become activated by mutation or are abnormally expressed in many human cancers. For example, the epidermal growth factor receptor (EGFR) is found mutated and/or overexpressed in breast, lung, brain, squamous cell, gastric, and other human cancers. Selective inhibitors of the tyrosine kinase activity of EGFR have been shown to be of clinical value in treatment of cancers with mutated and/or overexpressed EGFR. Thus, selective inhibitors of particular tyrosine kinases are useful in the treatment of proliferative diseases such as cancer.

FAK (encoded by the gene PTK2) is a non-receptor tyrosine kinase that integrates signals from integrins and growth factor receptors. FAK has been reported to play a role in the regulation of cell survival, growth, spreading, migration and invasion (McLean et al 2005, Nat Rev Cancer 5:505-515). Furthermore, FAK is regulated and activated by phosphorylation on multiple tyrosine residues. Overexpression of FAK mRNA and/or protein has been documented in many human tumors, including cancers of the breast, colon, thyroid, and prostate (Owens et al. 1995, Cancer Research 55: 2752-2755; Agochiya et al. 1999, Oncogene 18:5646-5653; Gabarro-Niecko et al. 2003, Cancer Metastasis Rev. 22:359-374). More significantly, there is evidence that phosphorylated FAK is increased in malignant compared to normal tissues (Grisaru-Granovsky et al. 2005, Int. J. Cancer 113: 372-378).

Inhibition of FAK by RNAi or expression of a FAK dominant negative has been shown to induce loss of adhesion and cell death in human breast and melanoma cell lines, and to augment docetaxel-mediated apoptosis in ovarian cancer cells (Beviglia et al 2003, Biochem J. 373:201-210, Smith et al 2005, Melanoma Res. 15:357-362, Halder et al 2005, Clin. Cancer Res. 11:8829-8836). However, inhibition of FAK in normal human fibroblasts or immortalized mammary cells (MCF10A) was found not to cause loss of attachment or apoptosis (Xu et al. 1996 Cell Growth and Diff 7:413-418). Inhibition of FAK by dominant negative expression has also been shown to reduce tumor growth and eliminate lung metastasis of mammary adenocarcinoma cells in a syngeneic rat model (van Nimwegen et al 2005, Cancer Res. 65:4698-4706). Similarly, inhibition of FAK by shRNA inhibited lung metastasis and reduced lethality by 40% in a syngeneic mouse model (Mitra et al 2006, Oncogene 25:4429-4440). In this study, transient re-expression of wild-type, but not kinase-dead FAK, reversed the shRNA phenotypes. Inhibition of FAK by dominant negative expression in mouse 4T1 carcinoma cells reduced tumor growth and angiogenesis in mice (Mitra et al 2006, Oncogene 25:5969-5984). Furthermore, loss of FAK catalytic activity (reconstitution of FAK−/− cells with kinase-dead FAK) reduced growth of v-Src tumors in mice and decreased angiogenesis.

Thus, there is strong evidence to suggest that inhibition of FAK activity induces apoptosis, loss of adhesion, inhibition of cell growth and migration, and that such inhibition reduces angiogenesis. Accordingly, compounds that inhibit FAK activity would be useful for the treatment of cancer.

### SUMMARY OF THE INVENTION

The present invention is a compound represented by formula (I):

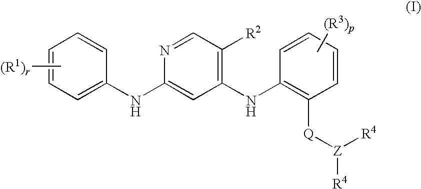

or a pharmaceutically acceptable salt thereof, wherein:
each R¹ is independently halo, hydroxy, cyano, nitro, —COOH, —COO—C₁-C₃-alkyl, C₁-C₆-alkoxy, —(NR⁵)ₓSO_{y}R⁶, —X—N(R⁷)₂, —SO_{y}N(R⁷)₂, —C₁-C₆-alkyl—(R⁸)ₚ, C₃-C₆-cycloalkyl-R⁹, phenyl—(R¹⁰)ₚ, heteroaryl—(R¹¹)ₚ, heterocycloalkyl-(R¹¹)ₚ, or two ortho R¹ groups, together with the carbon atoms to which they are attached, form a fused 5-6-membered carbocyclic or heterocyclic ring;
R² is halo, CF₃, C₁-C₆-alkyl, C₃-C₆-cycloalkyl, C₁-C₆-alkoxy, or cyano;
each R³ is independently halo, C₁-C₆-alkyl, —C₁-C₆-alkyl-R⁸, C₁-C₆-alkoxy, —X—N(R⁷)₂, or heterocycloalkyl;
each R⁴ is independently H, C₃-C₆-cycloalkyl, —C₁-C₆-alkyl-(R⁸)ₚ, hydroxy, or O—-C₁-C₆-alkyl-(R⁸)ₚ, or the R⁴ groups, together with Z, form a 5-6-membered cyclic ring optionally substituted with a —C₁-C₆-alkyl—(R⁸)ₚ group or a C₃-C₆-cycloalkyl group;
each R⁵ is independently H or —C₁-C₆-alkyl—(R⁸)ₚ;
each R⁶ is independently hydroxy, C₁-C₆-alkyl, phenyl—(R¹⁰)ₚ, or heteroaryl—(R¹⁰)ₚ;

each R⁷ is independently H, —(Y)ₓ—C₁-C₆-alkyl—R⁸, C₃-C₆-cycloalkyl, phenyl—(R¹⁰)ₚ, heteroaryl—(R¹⁰)ₚ, heterocycloalkyl—(R¹¹)ₚ, or, together with the nitrogen atom to which they are attached, form a 5-6-membered cyclic ring optionally substituted with a —C₁-C₆-alkyl—(R⁸)ₚ or C₃-C₆-cycloalkyl group;
each R⁸ is independently C₁-C₆-alkoxy, C₃-C₆-cycloalkyl, C₁-C₆-thioalkoxy, hydroxy, thiol, cyano, halo, nitro, —COOH, —COO—C₁-C₃-alkyl, —(NR⁵)ₓSO_{y}R⁶, —X—N(R⁷)₂, —SO_{y}N(R⁷)₂, phenyl—(R¹⁰)ₚ, or heteroaryl—(R¹⁰)ₚ;
each R⁹ is independently C₁-C₆-alkoxy, —COOH, —COO—C₁-C₃-alkyl, —X—N(R⁵)₂, halo, hydroxy group, or —C₁-C₆-alkyl—(R⁸)ₚ group;
each R¹⁰ is independently C₁-C₆-alkyl, C₁-C₆-alkoxy, hydroxy, halo, CF₃, or N(R⁵)₂;
each R¹¹ is independently C₁-C₆-alkyl-(R⁸)ₚ;
Q is —C(O)—, —S(O)—, or —SO₂—;
Z is N, CH, or C—C₁-C₆-alkyl;
X is a bond, —C₁-C₆-alkyl—, —C(O)—, or —O—(CH₂)_{q}—;
Y is —S(O)—, —SO₂—, —C(O)—, —C(O)NH—, or —C(O)O—;
each p is independently 0, 1, 2, or 3;
q is 1, 2, 3, or 4;
r is 0, 1, 2, or 3;
each x is independently 0 or 1; and
each y is independently 1 or 2.

In a further embodiment, the present invention relates to a composition comprising a) the compound of formula (I) or a pharmaceutically acceptable salt thereof; and b) a pharmaceutically acceptable excipient.

In a further embodiment, the present invention relates to a method of treating cancer comprising administering to a patient in need thereof a pharmaceutically effective amount of the compound of formula (I).

### DETAILED DESCRIPTION OF THE INVENTION

The present invention is a compound represented by formula (I):

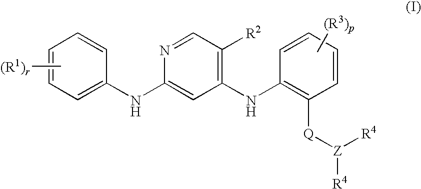

or a pharmaceutically acceptable salt thereof; where R¹-R⁴, Q, Z, r, and p are as defined herein.

As used herein, “halo” refers to fluoro, chloro, or bromo.

“C₁-C₆-alkyl” refers to a linear or branched alkyl group including methyl, ethyl, n-propyl, isopropyl, n-butyl, isobutyl, t-butyl, n-pentyl, and n-hexyl.

“C₁-C₆-alkoxy” refers to C₁-C₆-alkyl-O— groups, including methoxy, ethoxy, n-propoxy, iso-propoxy, and n-butoxy groups.

Examples of substituted C₁-C₆-alkyl groups (that is, —C₁-C₆-alkyl—(R⁸)ₚ, where p is 1, 2, or 3) include —CH₂CH₂OCH₃, —CH₂OCH₃, —CH₂OH, —CH₂CH₂OH, CF₃, —CH₂CF₃, —CH₂NHSO₂CH₃, —CH₂NHSOCH₃, —CH₂CH₂SO₂CH₃, —CH₂NH₂, —CH₂CH₂N(CH₃)₂, CH₂CH₂CH₂NH(CH₃), —CH₂C(O)N(CH₃)₂, and —CH₂OCH₂N(CH₃)₂.

C₃-C₆-cycloalkyl refers to a cyclopropyl, cyclobutyl, cyclopentyl, or cyclohexyl group.

As used herein, heterocycloalkyl refers to a 5- or 6-membered cycloaliphatic group that includes an O, N, or S heteroatom or a combination thereof. Suitable heterocycloalkyl groups include 1,3-dioxolanyl, 1,4-dioxolanyl, oxetanyl, pyranyl, tetrahydrofuranyl, pyrrolidinyl, pyrrolidinonyl, piperidinyl, piperazinyl, oxopiperazinyl, morpholino, and thiomorpholino groups.

The R⁴ (and R⁷) groups may, together with the nitrogen atom to which they are attached, form 5-6-membered cyclic ring optionally substituted with a —C₁-C₆-alkyl—R⁸ or C₃-C₆-cycloalkyl group. Suitable 5-6- membered cyclic rings include tetrahydrofuranyl, pyrrolidinyl, pyrrolidinonyl, piperidinyl, piperazinyl, oxopiperazinyl, morpholino, and thiomorpholino group.

Where r is 2 or 3, two ortho R¹ groups can, together with the carbon atoms to which they are attached, form a fused 5-6-membered carbocyclic or heterocyclic ring. As used herein, the term “ortho R¹ groups” means that the R¹ groups are ortho to each other. The formed ring may be aromatic, heteroaromatic, cycloaliphatic, or heterocycloaliphatic. Heteroaromatic and heterocycloaliphatic rings are 5-6-membered rings that include 1 to 3 heteroatoms selected from N, O, and S. Examples of fused heteroaromatic and heterocycloaliphatic ring groups include:

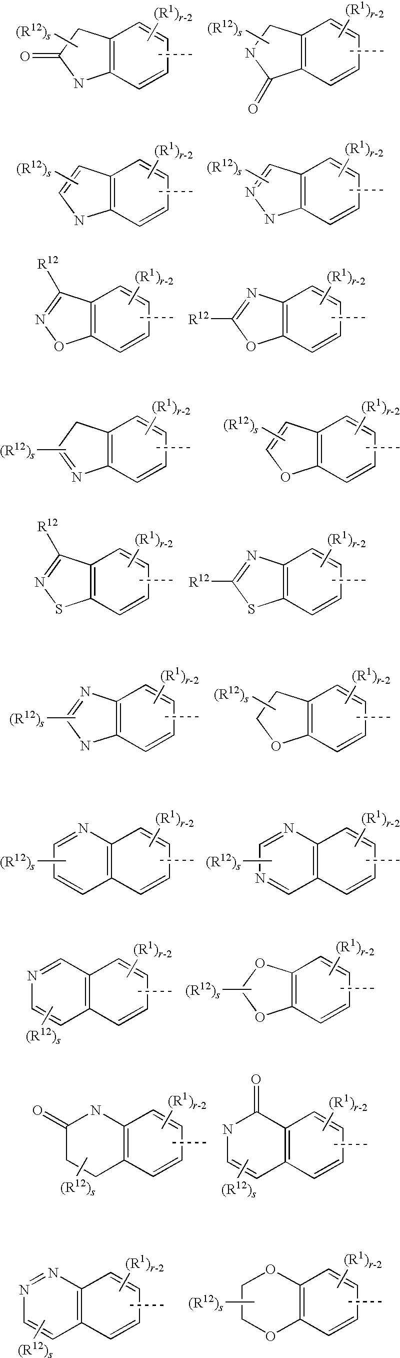

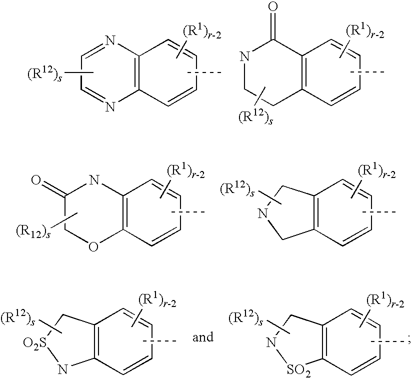

where r is 2 or 3; and each R¹² is independently H, F, CF₃, cyano, —(Y)ₓ-C₁-C₆-alkyl—R⁸, —C(O)—O—C₁-C₆-alkyl, C₃-C₆-cycloalkyl, —(NR⁵)ₓSO_{y}R⁶, —X—N(R⁷)₂, —SO_{y}N(R⁷)₂, phenyl—(R¹⁰)ₚ, heteroaryl—(R¹⁰)ₚ, or heterocycloalkyl—(R)¹¹)ₚ; where s is from 0 to up to the number of substitutable hydrogen atoms on the fused ring, not to exceed 3 substituents; and R⁵-R⁸, R¹⁰, R¹¹ and p are as previously defined. The dotted line indicates fusion at any two adjacent positions on the phenyl ring. Thus, the partial structure:

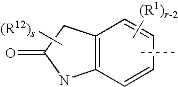

can have any of the following points of attachment:

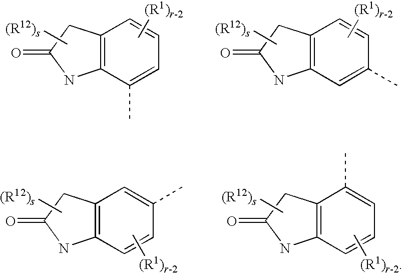

In another aspect, the present invention is a subclass of the compound of formula (I) represented by the following structure:

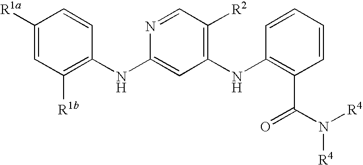

or a pharmaceutically acceptable salt thereof, wherein
R^{1a} is heterocycloalkyl—(R¹¹)ₚ, —C₁-C₆-alkyl-R⁸, or C₁-C₆-alkoxy;
R^{1b} is H, C₁-C₃ alkoxy, halo, CF₃, SO_{y}C₁-C₃-alkyl, or —C(O)N(R⁷)₂;
R² is halo or CF₃;
each R⁴ is independently H methyl, or methoxy; and
each R⁷ is independently H, C₁-C₃-alkyl, or, together with the nitrogen atom to which they are attached form a pyrrolidinyl, piperidinyl, piperazinyl, morpholino, or thiomorpholino group; wherein R^{1a} and R^{1b} are subgroups of R¹.

In another aspect, the present invention is a subclass of the compound of formula (I) represented by the following structure:

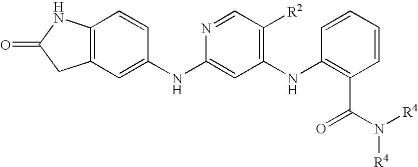

or a pharmaceutically acceptable salt thereof, wherein
R² is halo or CF₃; and
each R⁴ is independently H or methyl.

In another aspect, the present invention is a subclass of the compound of formula (I) represented by the following formula:

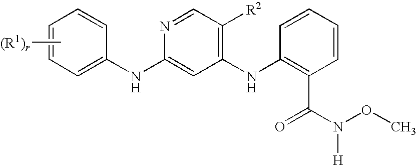

or a pharmaceutically acceptable salt thereof.

In another aspect, R² is halo or CF₃.

In another aspect, R^{1a} is a morpholino group and R^{1b} is a methoxy group.

As used herein, “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication.

The skilled artisan will appreciate that pharmaceutically acceptable salts of compounds according to formula (I) may be prepared. More particularly, inasmuch as compounds according to formula (I) contain a basic functional group—and may include an acid functional group—they are capable of forming pharmaceutically acceptable salts by treatment with a suitable acid or base. Suitable acids include pharmaceutically acceptable inorganic acids and organic acids. Representative pharmaceutically acceptable acids include hydrogen chloride, hydrogen bromide, nitric acid, sulfuric acid, sulfonic acid, phosphoric acid, acetic acid, hydroxyacetic acid, phenylacetic acid, propionic acid, butyric acid, valeric acid, maleic acid, acrylic acid, fumaric acid, malic acid, malonic acid, tartaric acid, citric acid, salicylic acid, benzoic acid, tannic acid, formic acid, stearic acid, lactic acid, ascorbic acid, p-toluenesulfonic acid, oleic acid, and lauric acid.

Suitable bases include pharmaceutically acceptable inorganic bases and organic bases. Representative pharmaceutically acceptable bases include sodium, potassium, iron, lithium, aluminium, calcium, zinc, arginine, choline, diethylenetriamine, dimethylamine, ethylenediamine, histidine, imidazole, lysine, morpholine, proline, trimethylamine, and tromethamine.

As used herein, the term “a compound of formula (I)” or “the compound of formula (I)” refers to one or more compounds according to formula (I). The compound of formula (I) may exist in solid or liquid form. In the solid state, it may exist in a crystalline or noncrystalline form, or as a mixture thereof. The skilled artisan will appreciate that pharmaceutically acceptable solvates may be formed for crystalline compounds wherein solvent molecules are incorporated into the crystalline lattice during crystallization. The incorporated solvent molecules may be water molecules or non-aqueous such as ethanol, isopropanol, DMSO, acetic acid, ethanolamine, and ethyl acetate molecules. Crystalline lattice incorporated with water molecules are typically referred to as “hydrates.” Hydrates include stoichiometric hydrates as well as compositions containing variable amounts of water. The present invention includes all such solvates.

The following schemes illustrate how compounds of the present invention can be prepared. The specific solvents and reaction conditions referred to are also illustrative and are not intended to be limiting.

### SCHEMES

Compounds of formula (I) can be prepared by methods outlined in the schemes below. R¹-R⁴, Q, Z, r and p are as previously defined and L¹ and L² are leaving groups such as halo, tosyl, or mesyl groups. Compounds of formula (II) and (III) are commercially available or readily preparable using techniques conventional in the art. Compounds of formula (II) and (III) may be coupled under Buchwald-Hartwig coupling conditions to form intermediate (IV). Alternatively, compounds of formula (II) and (III) can be coupled to form intermediate (IV) under addition reaction conditions, at a suitable temperature and in the presence of a suitable solvent, such as a polar, protic solvent. The coupling reaction is carried out under such conditions that leaving group L² is displaced selectively over leaving group L¹, or vice versa. When a compound of formula (II) includes a functional group in need of protection, for example, a hydroxyl or amino group, an appropriate protecting group is advantageously used prior to reaction with a compound of formula (III).

Intermediate (IV) can be coupled with aniline (V) (commercially available or preparable using techniques conventional in the art) under Buchwald-Hartwig coupling conditions to form a compound of formula (I). Alternatively, the coupling reaction can be carried out at a suitable temperature using an acid catalyst (such as 10-30 mol % hydrochloric acid or trifluoroacetic acid) in a suitable solvent (such as water, 1,4-dioxane, or iso-propanol).

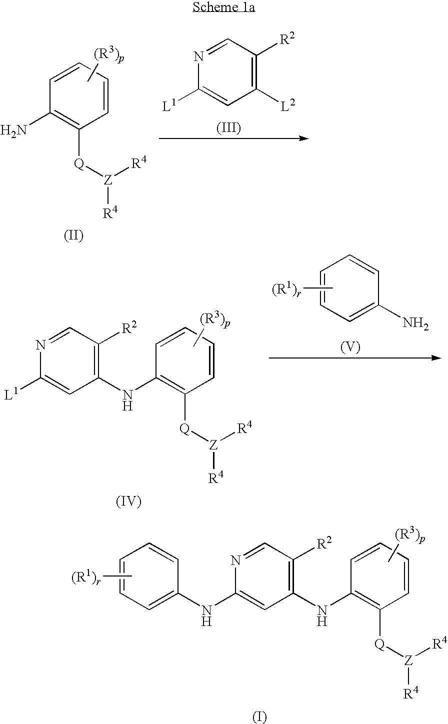

As shown in Scheme 1b compounds of formula (I) can be synthesized in a fashion where the order of coupling reactions has been reversed relative to Scheme 1.

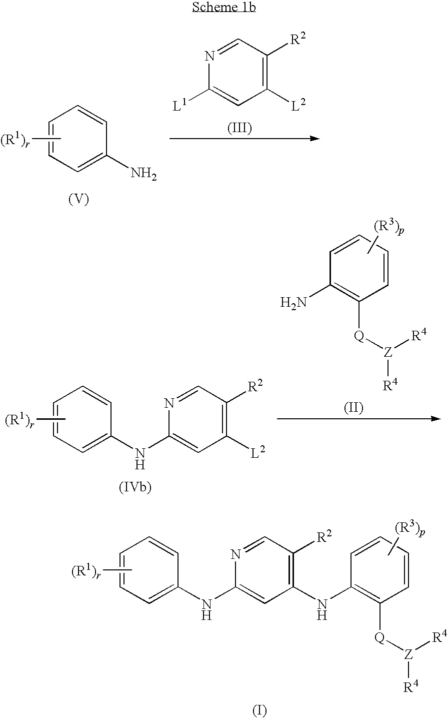

Scheme 2 illustrates a preparation of benzamide (VIII), which is a class of compounds of the present invention. Reaction of anthranilamide (VI) with pyridine (III) provides intermediate (VII), which then can be coupled with aniline (V) to provide a compound of formula (VIII).

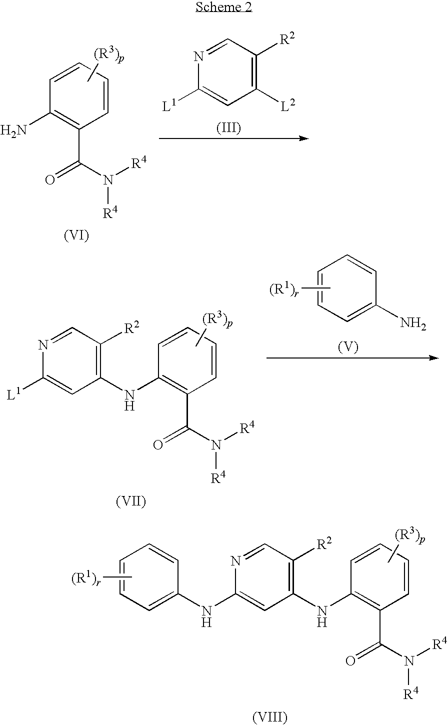

Compound (VI) may be prepared by reacting benzoxazine (IX) (commercially available or preparable using techniques conventional in the art) with an amine, as illustrated in Scheme 3.

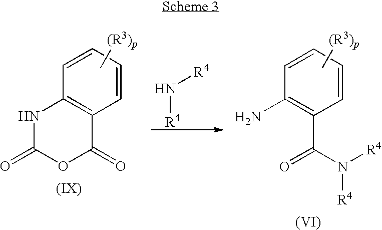

An alternative method for preparing a compound of formula (VIII) is shown in Scheme 4. Reaction of aminobenzoate (X) with pyridine (III) provides intermediate (XI), which then can be coupled with aniline (V) to provide intermediate (XII). Reaction of intermediate (XII) with an amine results in a compound of formula (VIII).

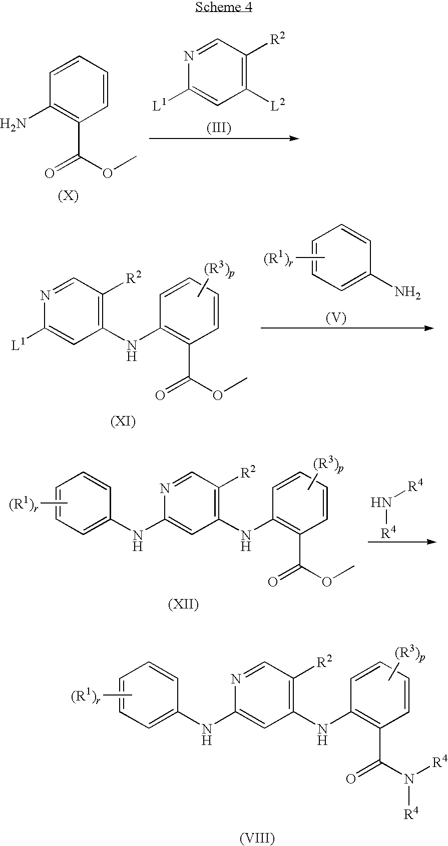

As shown in Scheme 5, a compound of formula (VIII) may also be prepared by coupling a compound of formula (VI) with a compound of formula (XIII) to form intermediate XIV as described for Scheme 1. Intermediate (XIV) may then be coupled with a compound of formula (XV) to provide a compound of formula (VIII). The reaction may be carried out in inert solvent, in the presence of a metal catalyst and appropriate ligand.

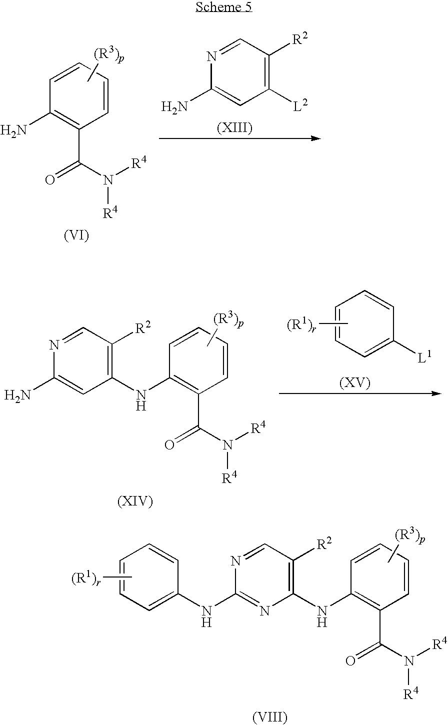

Compound of formula (XVIII) can be prepared by coupling compounds of formula (XVI) and formula (III) to form intermediate (XVII), which can be reacted with aniline (V) to give a compound of formula (XVIII).

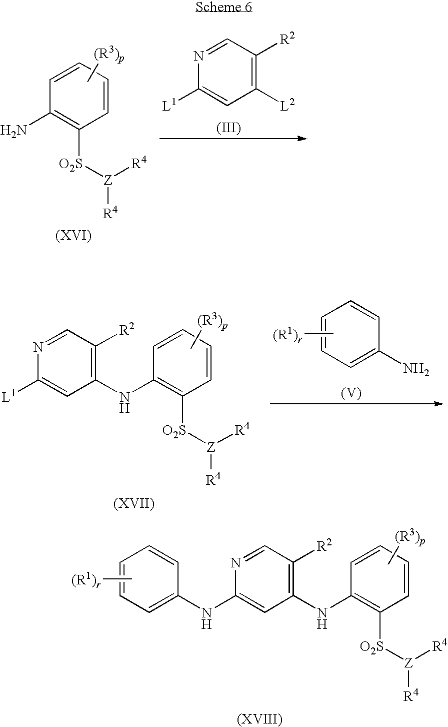

Sulfonamide (XXII) can be prepared by coupling compounds of formula (XIX) and formula (III) to form benzenesulfonic acid (XX). This intermediate can then be converted to the corresponding benzenesulfonyl chloride and aminated to form benzenesulfonamide (XXI), which can be coupled with aniline (V) to form the desired product.

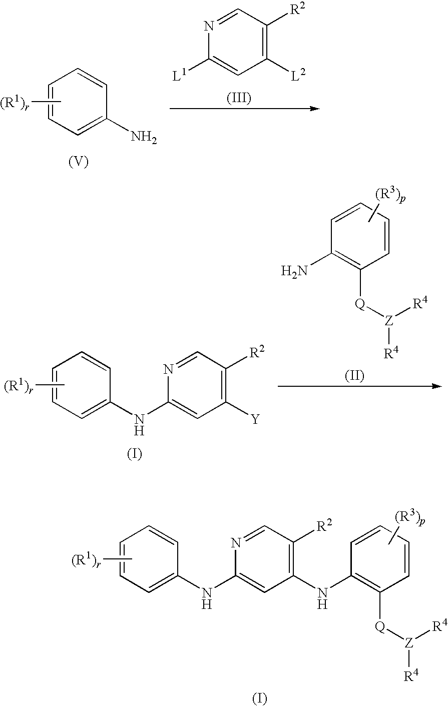

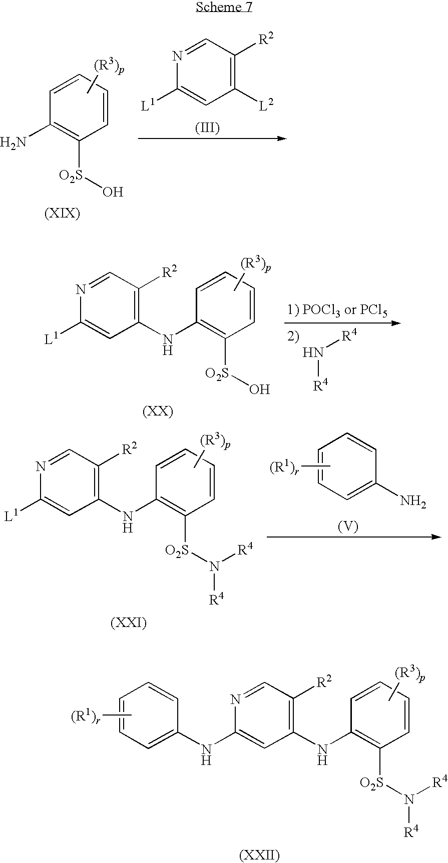

An alternative method for preparing a compound of formula (VIII) is shown in Scheme 8. Reaction of aminobenzoic acid (XXIII) with pyridine (III) provides intermediate (XXIV), which then can be coupled with aniline (V) to provide intermediate (XXV). Reaction of intermediate (XXV) with an amine results in a compound of formula (VIII).

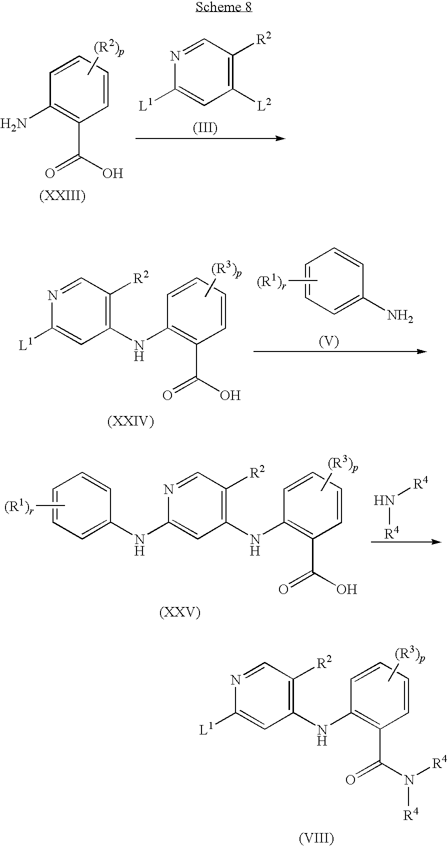

### EXPERIMENTAL

Biochemical assay for FAK activity

GST-tagged FAK was purchased from Invitrogen (PV3832). The activity of FAK was measured by monitoring the phosphorylation of a peptide substrate

(Ac-RRRRRRSETDDYAEIID-NH₂) in the presence of a radio-labeled ATP. To measure inhibitors of FAK, compounds were first prepared as a 10× stock in 10% DMSO. A small portion of each solution (4 μL) was added to a 96-well plate (Corning, 3884). A 6-nM GST-FAK solution was prepared in 1.1 × reaction buffer containing 44 mM HEPES, pH=7.2, 11 mM MgCl₂, 2.2 mM MnCl₂, 1.1 mM DTT and 0.011% Tween-20. Then, 20 μL of the 6 nM GST-FAK solution were pre-incubated with the compounds for 30 min at room temperature. The reaction was initiated by adding 16 μL of substrates (62.5 μM peptide, 5 μM ATP and ˜0.02 mCi/ml³³P-γ-ATP) prepared in the above reaction buffer. The reaction was allowed to proceed for 90 min before being quenched with 40 μL of 1% H₃PO₄. A portion of the reaction mixture (60 μL) was transferred to a phospho-cellulose filter plate (Millipore, MAPHNOB50) and incubated for 20 minutes. The plate was filtrated, washed three times using 150 μL of 0.5% H₃PO₄ and dried at 50° C. for 30 min. After the addition of 60 μL Microscint-20 to the plate, radioactivity was measured using a TopCount (PerkinElmer).

### EXAMPLES

The following examples are for illustrative purposes only and are not intended to limit the scope of the present invention. The compounds were named using ACD Name software (Advanced Chemistry Development). All compounds have pIC₅₀ of greater than 7 for FAK. A PE Sciex API 150 single quadrupole mass spectrometer (PE Sciex, Thornhill, Ontario, Canada) was operated using electrospray ionization in the positive ion detection mode. The nebulizing gas was generated from a zero air generator (Balston Inc., Haverhill, Mass.) and delivered at 65 psi and the curtain gas was high purity nitrogen delivered from a Dewar liquid nitrogen vessel at 50 psi. The voltage applied to the electrospray needle was 4.8 kV. The orifice was set at 25 V and mass spectrometer was scanned at a rate of 0.5 scan/sec using a step mass of 0.2 amu and collecting profile data.

Samples are introduced into the mass spectrometer using a CTC PAL autosampler (LEAP Technologies, Carrboro, N.C.) equipped with a hamilton 10 uL syringe which performed the injection into a Valco 10-port injection valve. The HPLC pump was a Shimadzu LC-10ADvp (Shimadzu Scientific Instruments, Columbia, Md.) operated at 0.3 mL/min and a linear gradient 4.5% A to 90% B in 3.2 min. with a 0.4 min. hold. The mobile phase was composed of 100% (H2O 0.02% TFA) in vessel A and 100% (CH3CN 0.018% TFA) in vessel B. The stationary phase is Aquasil (C18) and the column dimensions are 1mm×40 mm. Detection was by UV at 214 nm, evaporative light-scattering (ELSD) and MS.

Alternatively, an Agilent 1100 analytical HPLC system with an LC/MS was used and operated at 1 mL/min and a linear gradient 5% A to 100% B in 2.2 min with a 0.4 min hold. The mobile phase was composed of 100% (H₂O 0.02% TFA) in vessel A and 100% (CH₃CN 0.02% TFA) in vessel B. The stationary phase was Zorbax (C8) with a 3.5 um particle size and the column dimensions were 2.1 mm×50 mm. Detection was by UV at 214 nm, evaporative light-scattering (ELSD) and MS.

¹H-NMR (hereinafter “NMR”) spectra were recorded at 400 MHz using a Bruker AVANCE 400 MHz instrument, with ACD Spect manager ver. 10 using for reprocessing. Multiplicities indicated are: s=singlet, d=doublet, t=triplet, q=quartet, m=multiplet, dd=doublet of doublets, dt=doublet of triplets etc. and br indicates a broad signal.

Analytical HPLC: Products were analyzed by Agilent 1100 Analytical Chromatography system, with 4.5×75 mm Zorbax XDB-C18 column (3.5 μm) at 2 mL/min with a 4 min gradient from 5% CH₃CN (0.1% formic acid) to 95% CH₃CN (0.1% formic acid) in H₂O (0.1% formic acid) and a 1 min hold.

Preparative HPLC: Products were purified using a Gilson preparative chromatography system with a 75×30 mm I. D. YMC CombiPrep ODS-A column (5 μm) at 50 mL/min with a 10 min gradient from 5% CH₃CN (0.1% formic acid) to 95% CH₃CN (0.1% formic acid) in H₂O (0.1% formic acid) and a 2 min hold; alternatively, products were purified using an Agilent 1100 Preparative Chromatography system, with 100×30 mm Gemini C18 column (5 μm) at 60 mL/min with a 10 min gradient from 5% CH₃CN (0.1% formic acid) to 95% CH₃CN (0.1% formic acid) in H₂O (0.1% formic acid) and a 2 min hold.

Preparative normal phase chromatography was carried out using an Analogix IntelliFlash 280 System with SuperFlash Sepra Si 50 columns.

### Intermediate 1

2-{[2-Chloro-5-(trifluoromethyl)-4-pyridinyl]amino}-N-methylbenzamide:

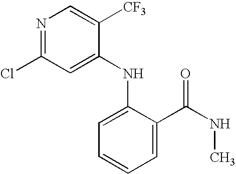

A vessel was charged with 2-chloro-4-iodo-5-(trifluoromethyl)pyridine (500 mg, 1.626 mmol), 2-amino-N-methylbenzamide (269 mg, 1.789 mmol), cesium carbonate (2649 mg, 8.13 mmol), and 10 mL of toluene, and degassed with nitrogen. (±)-2,2′-Bis(diphenylphosphino)-1,1′-binaphthalene (81 mg, 0.130 mmol) and palladium acetate (7.30 mg, 0.033 mmol) were added to the reaction mixture and the vessel was sealed and heated at 120° C. until complete. After the reaction was complete, the resulting suspension was cooled to room temperature and filtered through celite. The filtrate was evaporated to dryness and the crude mixture was purified by flash chromatography on a silica gel column eluting with ethyl acetate in hexane to afford the title compound (100 mg, 19% yield). MS: M(C₁₄H₁₁ClF₃N₃O)=329.71, (M+H)⁺=330.

### Intermediate 2

4-Iodo-5-methyl-N-[4-(4-morpholinyl)phenyl]-2-pyridinamine

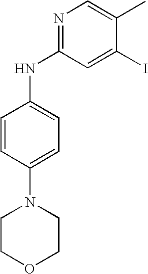

A vessel was charged with 2-fluoro-4-iodo-5-methylpyridine (1.0 g, 4.22 mmol), 4-morpholinoaniline (0.752 g, 4.22 mmol), 1.6 M hydrochloric acid (2.64 mL, 4.22 mmol), dioxane (5 mL), and water (5 mL), then sealed and heated at 120° C. for 16 h. The reaction mixture was then partitioned between saturated NaHCO₃ and ethyl acetate, and the organic phase was isolated, washed with brine and dried over MgSO₄. The final crude oil was triturated with ethyl acetate to afford the title compound as a solid (850 mg, 47% yield). MS: M(C₁₆H₁₈1N₃O)=395.24, (M+H)⁺=396.

### Intermediate 3

4-Iodo-5-methyl-N-[-2-(methyloxy)-4-(4-morpholinyl)phenyl]-2-pyridinamine

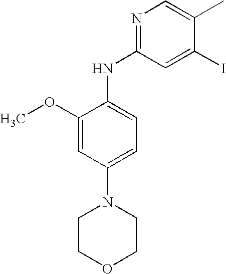

A vessel was charged with 2-fluoro-4-iodo-5-methylpyridine (1.0 g, 4.22 mmol), 2-(methyloxy)-4-(4-morpholinyl)aniline (500 mg, 2.043 mmol), 12 M hydrochloric acid (0.34 mL, 4.09 mmol), dioxane (5 mL), and water (5 mL), then sealed and heated at 120° C. for 24 h. The reaction mixture was then partitioned between saturated NaHCO₃ and ethyl acetate. The organic phase was isolated, then washed with brine and dried over MgSO₄. The crude oil was triturated with ethyl acetate to give the title compound as a solid (180 mg, 20% yield). MS: M(C₁₇H₂₀1N₃O₂)=425.26, (M+H)⁺=426.

### Intermediate 4

5-Chloro-4-iodo-N-[-4-(4-morpholinyl)phenyl]-2-pyridinamine

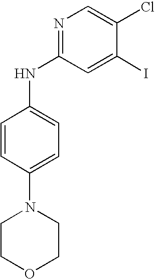

A vessel was charged with 5-chloro-2-fluoro-4-iodopyridine (794 mg, 3.09 mmol), 4-morpholinoaniline (0.5 g, 2.81 mmol), 4.0 M hydrochloric acid (1.4 mL, 5.61 mmol), dioxane (2.5 mL), and water (2.5 mL), and sealed and heated in 125° C. overnight. The reaction mixture was then partitioned between saturated NaHCO₃ and ethyl acetate and the organic phase was isolated, washed with brine and dried over MgSO₄. The final product was purified by flash chromatography on silica gel column eluting with ethyl acetate and hexane to give the title compound as a solid (200 mg, 16% yield). MS: M(C₁₅H₁₅ClIN₃O)=415.66, (M+H)⁺=417.

### Intermediate 5

5-Chloro-4-iodo-N-[2-(methyloxy)-4-(4-morpholinyl)phenyl]-2-pyridinamine

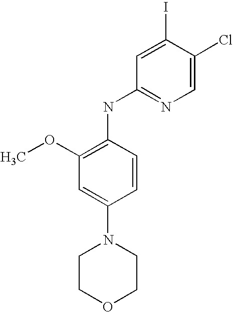

A vessel was charged with 5-chloro-2-fluoro-4-iodopyridine (579 mg, 2.247 mmol), 2-(methyloxy)-4-(4-morpholinyl)aniline (500 mg, 2.043 mmol), 1.0 M hydrochloric acid (4.09 mL, 4.09 mmol), dioxane (5 mL), and water (5 mL). The vessel was sealed and heated in a 85° C. oil bath for 36h. The reaction mixture was then partitioned between saturated NaHCO₃ and ethyl acetate. The organic phase was washed with brine and dried over MgSO₄. The crude mixture was purified by flash chromatography on silica gel column eluting with ethyl acetate in hexane to afford the title compound (182 mg, 18% yield). MS: M(C₁₆H₁₇ClN₃O₂)=445.68, (M+H)⁺=446.

### Intermediate 6

2-[(2-Fluoro-5-methyl-4-pyridinyl)amino]-N-methylbenzamide

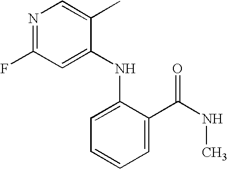

A microwave tube was charged with 2-fluoro-4-iodo-5-methylpyridine (47.4 mg, 0.2 mmol) and 2-amino-N-methylbenzamide (36.0 mg, 0.240 mmol), cesium carbonate (130 mg, 0.4 mmol), and 2.5 mL of toluene and charged with nitrogen. (±)-2,2′-Bis(diphenylphosphino)-1,1′-binaphthalene (4.98 mg, 8.00 μmol) and palladium acetate (0.898 mg, 4.00 μmol) were added to the reaction mixture. The microwave tube was sealed and heated at 100° C. for 16 h. The resulting suspension was cooled to room temperature and filtered through celite. The filtrate was evaporated to dryness and the crude reaction mixture was purified by flash chromatography on silica gel column eluting with ethyl acetate in hexane to give the title compound (23 mg, 41% yield). MS: M(C₁₄H₁₄FN₃O)=259.28, (M+H)⁺=260.

### Intermediate 7

2-[(2-Chloro-5-fluoro-4-pyridinyl)amino]-N-methylbenzamide

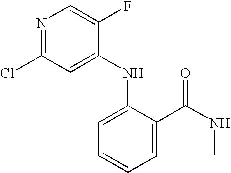

A microwave vessel was charged with 2-chloro-5-fluoro-4-iodopyridine (200 mg, 0.777 mmol), 2-amino-N-methylbenzamide (117 mg, 0.777 mmol), cesium carbonate (759 mg, 2.331 mmol) in 1,4-dioxane (2.5 mL). The reaction mixture was bubbled with argon for 2 min. The reaction mixture was charged with palladium(II) acetate (8.72 mg, 0.039 mmol) and (±)-2,2′-Bis(diphenylphosphino)-1,1′-binaphthalene (48.4 mg, 0.078 mmol). The reaction mixture was irradiated in a microwave oven at 180° C. for 20 min. The reaction mixture was reduced to dryness in the presence of silica gel (300 mg) and the resulting crude product was added to a silica gel column and eluted with hexanes and EtOAc (5% to 100%) to afford the title compound (54 mg, 25%). 1H NMR (400 MHz, DMSO-d₆) δ ppm 10.35 (d, J=2.02 Hz, 1 H), 8.71 (d, J=4.29 Hz, 1 H), 8.23 (d, J=3.03 Hz, 1 H), 7.72 (dd, J=7.83, 1.26 Hz, 1 H), 7.69−7.50 (m, 2 H), 7.23−7.17 (m, 2 H), 2.77 (d, J=4.55 Hz, 3 H).

### Intermediate 8

2-[(5-Bromo-2-chloro-4-pyridinyl)amino]-N-methylbenzamide

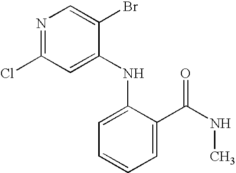

2-Amino-N-methylbenzamide (1 g, 6.66 mmol) and 5-bromo-2-chloro-4-iodopyridine (2.120 g, 6.66 mmol) were combined in a 20 mL reaction vessel with isopropanol (50 mL). 6 M Hydrochloric acid (1.110 mL, 6.66 mmol) was added and the vessel was sealed then heated at 95° C. for 96 h. The reaction mixture was evaporated to dryness and treated with ethyl acetate (20 mL) and 1 M Na₂CO₃ (10 mL). The organic layer was separated and evaporated. The residue was purified via normal-phase chromotography (15% ethyl acetate in dichloromethane) to give the title compound (205 mg, 7.76% yield). The solid was dissolved in dichloromethane and 1 mL of 1 M HCl in diethyl ether was added. The solid was filtered and 205 mg of the title compound was obtained as a white, hydrochloride salt. 1H NMR (400 MHz, DMSO-d₆) δ ppm 2.77 (d, J=4.55 Hz, 3 H) 5.07 (br. s., 5 H) 7.17 (s, 1 H) 7.18-7.25 (m, 1 H) 7.50-7.58 (m, 1 H) 7.58-7.64 (m, 1 H) 7.74 (dd, J=7.83, 1.26 Hz, 1 H) 8.38 (s, 1 H) 8.77 (d, J=4.55 Hz, 1 H) 10.34 (s, 1 H).

### Intermediate 9

2-[(5-Chloro-2-{[2-(methyloxy)-4-(4-morpholinyl)phenyl]amino}-4-pyridinyl)amino]benzoic acid

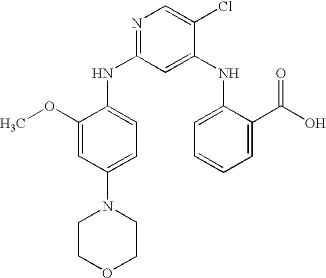

A sealable tube was charged with 2-[(2,5-dichloro-4-pyridinyl)amino]benzoic acid (600 mg, 2.119 mmol), 2-(methyloxy)-4-(4-morpholinyl)aniline (485 mg, 2.331 mmol), BINAP (198 mg, 0.318 mmol), Pd2(dba)₃ (97 mg, 0.106 mmol) and sodium tert-butoxide (509 mg, 5.30 mmol) in 1,4-dioxane (30 mL). The reaction mixture was degassed with nitrogen, sealed and heated in oil bath at 180° C. for 72 hours. The reaction mixture was cooled and then partitioned between saturated NaHCO₃ and ethyl acetate. The organic phase was washed with brine and dried over by MgSO₄. The organic solvent was evaporated under vacuum to give the title compound with 94% purity.

### Intermediate 10

### 2-Amino-N-methoxy-benzamide

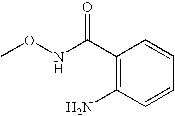

To a mixture of isatoic anhydride (40 g, 245.39 mmol, 1 eq) and o-methyl hyroxylamine hydrochloride (30.55 g, 368.09 mmole, 1.5 eq) in EtOH:H₂O (9:1) (1000 mL) was added TEA (51.2 mL, 368.09 mmole, 1.5 eq) and resulting mixture was reflux for 4 h. After completion of reaction solvent was removed under reduced pressure and residue was diluted with water (500 mL), extracted with ethyl acetate (3×250 mL). Combined organic layer was dried over sodium sulfate, filtered and concentrated under reduced pressure. Solid compound so obtained was purified by washing with diethyl ether and hexane to give the title compound as a brown solid (20 g, 49%). ¹H-NMR (400 MHz, DMSO-d₆): δ 3.67 (s, 3H), 6.20-6.40 (brs, 2H), 6.44-6.53 (m, 1H), 6.70 (d, 1H, J=7.76 Hz), 7.10-7.19 (m, 1H), 7.30 (d, 1H, J=7.6 Hz), 11.40 (s, 1H). LC-MS calculated for C₈H₁₀N₂O₂ [M+H] 167.07, found 167.2. HPLC purity 99.60% at 200 nm.

### Intermediate 11

2-(5-bromo-2-chloro-pyridin-4-ylamino)-N-methoxy-benzamide

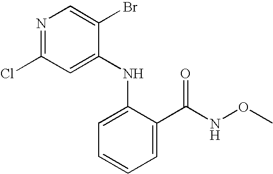

A mixture of 5-bromo-2-chloro-4-iodo-pyridine (5 g, 15.72 mmole, 1 eq), 2-amino-N-methoxy-benzamide (2.61 g, 15.72 mmole, 1 eq) and K₃PO₄ (8.34 g, 39.3 mmole, 2.5 eq), and 1,4-dioxane (30 mL) was degassed with N₂ for 1 h. To this DPEphos (0.67 g, 1.25 mmole, 0.08 eq) and Pd(OAc)₂ (0.07 g, 0.31 mmole, 0.02 eq) were added and degassed again with N₂ for 30 min. The resulting mixture was refluxed for overnight. After completion of reaction, solvent was removed under reduced pressure and residue was diluted with water (100 mL) and extracted with 5% MeOH-DCM (3×100 mL). Combined organic layer was dried over sodium sulfate, filtered and concentrated under reduced pressure to give crude compound. Crude compound was purified by column chromatography over silica gel (60-120 mesh) using 20% ethyl acetate-hexane as eluant to yield title compound as off white solid (3.5 g, 62%). ¹H-NMR (400 MHz, DMSO-d₆): δ 3.56 (s, 3H), 6.88-6.98 (m, 1H), 7.08 (s, 1H), 7.17-7.25 (m, 1H), 7.26-7.35 (m, 1H), 7.82-7.90 (m, 1H), 8.23 (s, 1H), 12.86 (brs, 1H). LC-MS calculated for C₁₃H₁₁BrClN₃O₂⁻; [M+H] 355.97, found 356.30.

### Intermediate 12

2-(2-Chloro-5-cyclopropyl-pyridin-4-ylamino)-N-methoxy-benzamide

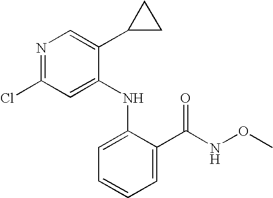

In a sealable tube toluene (50 mL) was degassed with N₂ at 50° C. for 15 min and to this 2-(5-bromo-2-chloro-pyridin-4-ylamino)-N-methoxy-benzamide (1.5 g, 4.21 mmole, 1 eq), cyclopropylboronic acid (1.4 g, 16.85 mmole, 4 eq) and Pd(PPh₃)₄ (0.24 g, 0.21 mmole, 0.05 eq) were added and resulting mixture was degassed for 30 min. To this NaBr (0.44 g, 4.33 mmole, 1.03 eq) and a solution of KF (0.8 g, 13.90 mmole, 3.3 eq) in H₂O (3 mL) were added; again degassed with N₂ for 15 min. The tube was sealed and the mixture was heated at 100° C. for 24 h. After completion of reaction, reaction mixture was allowed to cool at room temperature, poured into water (100 mL) and extracted with toluene (2×50 mL). Combined organic layer was dried over sodium sulfate, filtered and concentrated under reduced pressure. The crude compound was purified by washing with 0.5% DCM-Et₂O to give title compound as pale yellow solid (0.7 g, 53%). ¹H-NMR (400 MHz, DMSO-d₆): δ 0.57-072 (m, 2H), 0.97-1.10 (m, 2H), 1.62-1.75 (m, 1H), 3.68 (s, 3H), 7.02 (s, 1H), 7.11-7.20 (m, 1H), 7.51-7.67 (m, 3H), 7.94 (s, 1H), 9.62 (s, 1H), 11.92 (brs, 1H). LC-MS calculated for C₁₆H₁₆ClN₃O₂ [M+H] 318.09, found 318.2.

### Intermediate 13

2-(2-Chloro-5-cyclopropyl-pyridin-4-ylamino)-N-methyl-benzamide

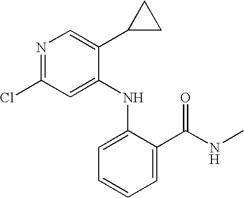

A solution of cyclopropylboronic acid (0.38 g, 4.40 mmole, 1.5 eq) in toluene (100 mL) was degassed with N₂ at 50° C. for 15 min and to this Pd(PPh₃)₄ (0.17 g, 0.15 mmole, 0.05 eq) and 2-(5-bromo-2-chloro-pyridin-4-ylamino)-N-methyl-benzamide (1 g, 2.93 mmole, 1 eq) was added and resulting reaction mixture was again degassed for 30 min. To this a degassed solution of K₃PO₄ (2.49 g, 11.72 mmole, 4 eq) in H₂O (4 mL) was added in one portion and resulting reaction mixture was refluxed for overnight. After completion of reaction, solvent was removed under reduced pressure and residue was diluted with water (100 mL) and then extracted with DCM (3×75 mL). Combined organic layer was dried over sodium sulfate, filtered and concentrated under reduced pressure to yield crude product. Crude compound was purified by column chromatography over silica gel (60-120 mesh) using 0.5% MeOH-DCM as eluant to give title compound as a pale yellow solid (0.480 g, 54%). ¹H-NMR (400 MHz, DMSO-d₆): δ 0.60-0.70 (m, 2H), 0.95-1.05 (m, 2H), 1.60-1.71 (m, 1H), 2.76 (d, 3H, J=4.48 Hz), 7.09 (s, 1H), 7.10-7.18 (m, 1H), 7.49-7.60 (m, 2H), 7.68-7.70 (d, 1H, J=7.64 Hz), 7.93 (s, 1H), 8.60-8.70 (m, 1H), 10.22 (s, 1H). LC-MS calculated for C₁₆H₁₆ClN₃O [M+H] 302.10, found 302.0.

### Intermediate 14

2-(2-Chloro-5-vinyl-pyridin-4-ylamino)-N-methyl-benzamide

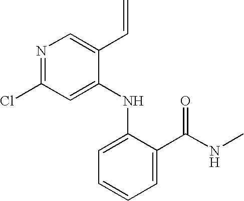

A solution of vinylboronic acid pinacol ester (2.25 mL, 13.2 mmole, 1.5 eq) in toluene (150 mL) was degassed with N₂ for 15 min and to this was added Pd(PPh₃)₄ (0.5 g, 0.44 mmole, 0.05 eq) followed by 2-(5-bromo-2-chloro-pyridin-4-ylamino)-N-methyl-benzamide (3 g, 8.8 mmole, 1 eq) and again degassed with N₂ at 50° C. for 15 min. To this mixture a degassed solution of K₃PO₄ (7.5 g, 35.23 mmole, 4 eq) in H₂O (9 mL) was added in one portion and resulting mixture was refluxed for overnight. After completion, reaction mixture was allowed to cool to room temperature, diluted with water (100 mL) and extracted with ethyl acetate (3×100 mL). Combined organic layer was dried over sodium sulfate, filtered and concentrated under reduced pressure to give a crude product. Crude compound was purified by column chromatography over silica gel (60-120 mesh) using 1% MeOH-DCM as eluant to give the title compound as an off white solid (2 g, 80%). ¹H-NMR (400 MHz, DMSO-d₆): δ 2.74 (d, 3H, J=4.52 Hz), 5.50-5.60 (m, 1H), 5.84-5.94 (m, 1H), 6.70-6.83 (m, 1H), 7.09 (s, 1H), 7.11-7.19 (m, 1H), 7.45-7.58 (m, 2H), 7.69-7.71 (d, 1H, J=7.64 Hz), 8.26 (s, 1H), 8.60-8.78 (brs, 1H), 10.18 (s, 1H). LC-MS calculated for C₁₅H₁₄ClN₃O [M+H] 288.08, found 288.2.

### Intermediate 15

6-Chloro-4-iodo-nicotinonitrile

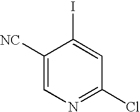

To a mixture of THF (100 mL) and Hexane (40 mL) under nitrogen atmosphere was added DIPA (22.21 mL, 158.78 mmole, 1.1 eq), the mixture was allowed to cool at −80° C. and to this n-BuLi (63.57 mL, 158.78 mmole, 1.1 eq) was added dropwise. After completion of addition the resulting mixture was allowed to warm and stirred at −10° C. for 15 min. Reaction mixture was again cooled at −80° C. and solution of 6-chloro-nicotinonitrile (20 g, 144.35 mmol, 1 eq) in THF (100 mL) was added dropwise. The resulting mixture stirred at −80° C. for 1 h. After 1 h a solution of Iodine (43.96 g, 173.22 mmole, 1.2 eq) in THF (100 mL) was added in one portion. After completion, reaction mixture was quenched with water (100 mL) and extracted with diethyl ether (6×100 mL). Combined organic layer was washed with saturated solution of sodium thosulfate (2×100 mL), dried over sodium sulfate, filtered and concentrated under vacuum to give crude product. Crude compound was purified by column chromatography over silica gel (100-200 mesh) using 2% ethyl acetate-hexane as eluant to yield the title compound as a faint yellow solid (15 g, 39%). ¹H-NMR (400 MHz, DMSO-d₆): δ 8.38 (s, 1H), 8.79 (s, 1H). LC-MS calculated for C₆H₂ClIN₂ (M+H) 264.90, found 264.9. HPLC purity 96.0% at 220 nm.

### Intermediate 16

2-(2-Chloro-5-cyano-pyridin-4-ylamino)-N-methyl-benzamide

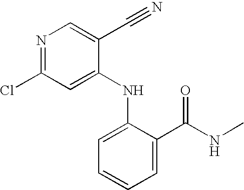

A mixture of 6-chloro-4-iodo-nicotinonitrile (14 g, 53.03 mmole, 1 eq), 2-amino-N-methoxy-benzamide (7.96 g, 53.03 mmole, 1 eq) and K₃PO₄ (28.14 g, 132.57 mmole, 2.5 eq) in 1,4-dioxane (250 mL) was degassed with N₂ for 1 h. To this mixture were added Pd(OAC)₂ (0.238 g, 1.06 mmole, 0.02 eq) and DPEphos (2.28 g, 4.24 mmole, 0.08 eq) and resulting reaction mixture was degassed with N₂ for another 15 min and resulting reaction mixture was stirred at 110° C. for overnight. After completion of reaction solid material were collected by filtration, dissolved in water (500 mL) and extracted with ethyl acetate (5×200 mL). Combined organic layer was dried over sodium sulfate, filtered and concentrated under reduced pressure to give crude compound. Crude compound was purified by column chromatography over silica gel (60-120 mesh) using 0.2% methanolic ammonia (10% ammonia in MeOH) in dichloromethane as eluant to give the title compound as a pale yellow solid (9 g, 59%). ¹H-NMR (400 MHz, DMSO-d₆): δ 2.75 (d, 3H, J=4.56 Hz), 7.09 (s, 1H), 7.25-7.35 (m, 1H), 7.53-7.60 (m, 2H), 7.72 (d, 1H, J=7.48 Hz), 8.56 (s, 1H), 8.69-8.79 (brs, 1H), 10.65 (s, 1H). LC-MS calculated for C₁₄H₁₁ClN₄O (M+H) 287.06, found 286.9. HPLC purity 99.57% at 200 nm.

### Example 1

N-Methyl-2-{[2-[2-oxo-2.3-dihydro-1H-indol-5-yl)amino]-5-(trifluoromethyl)-4-pyridinyl]amino}benzamide

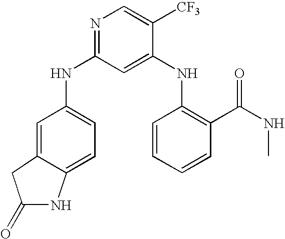

2-{[2-Chloro-5-(trifluoromethyl)-4-pyridinyl]amino}-N-methylbenzamide (Intermediate 1, 50 mg, 0.152 mmol), 5-amino-1,3-dihydro-2H-indol-2-one (90 mg, 0.607 mmol), 1.0 M hydrochloric acid (0.3 mL) 1,4-dioxane (1.0 mL), and water (1.0 mL) were added to a vessel, which was sealed and heated at 150° C. for 2 days. The resulting crude material was purified by reverse-phase HPLC to give the title compound as a solid (20 mg, 26% yield). MS: M(C₂₂H₁₈F₃N₅O₂)=441.41, (M+H)⁺=442; 1H NMR (400 MHz, DMSO-d6) δ ppm 2.76 (d, J=4.55 Hz, 3 H) 3.45 (s, 2 H) 6.68 (s, 1 H) 6.74 (d, J=8.34 Hz, 1 H) 7.11 (dd, J=15.03, 1.14 Hz, 1H) 7.26 (dd, J=8.46, 2.15 Hz, 1 H) 7.43-7.52 (m, 1 H) 7.54-7.60 (m, 1 H) 7.71 (dd, J=7.83, 1.26 Hz, 1 H) 8.18 (s, 1 H) 8.23 (s, 1 H) 8.67 (d, J=4.55 Hz, 1H) 9.07(s, 1 H) 10.15 (s, 1 H) 10.24 (s, 1 H).

### Example 2

N-Methyl-2-{[2-{[4-(4-morpholinyl)phenyl]amino}-5-(trifluoromethyl)-4-pyridinyl]amino}benzamide

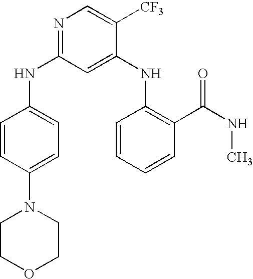

A microwave tube was charged with 2-{[2-chloro-5-(trifluoromethyl)-4-pyridinyl]amino}-N-methylbenzamide (Intermediate 1, 60 mg, 0.182 mmol), 4-(4-morpholinyl)aniline (39.0 mg, 0.219 mmol) and 0.4 mL of 1.0 M hydrochloric acid in 2.5 mL of 1,4-dioxane, then irradiated in a microwave oven at 160° C. for 1 hour. The resulting crude material was purified using reverse-phase HPLC to give the title compound (23 mg, 23% yield). MS: M(C₂₃H₂₄F₃N₅O₂)=471.48, (M+H)⁺=472; 1H NMR (400 MHz, DMSO-d6) δ ppm 2.76 (d, J=4.55 Hz, 3 H) 3.01-3.03 (m, 4 H) 3.72-3.74 (m, 4 H) 6.67 (s, 1 H) 6.88 (d, J=8.84 Hz, 2H) 7.10 (dd, J=15.03, 1.14 Hz, 1H) 7.41 (m, J=9.09 Hz, 2 H) 7.47-7.61 (m, 2 H) 7.70 (dd, J=7.83, 1.52 Hz, 1 H) 8.22 (s, 1 H) 8.67 (d, J=4.55 Hz, 1 H) 9.01 (s, 1 H) 10.15 (s, 1 H).

### Example 3

N-Methyl-2-{[2-{[-2-(methyloxy)-4-(4-morpholinyl)phenyl]amino}-5-(trifluoromethyl)-4-pyridinyl]amino}benzamide

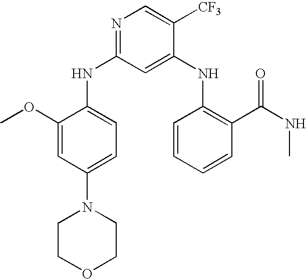

2-{[2-Chloro-5-(trifluoromethyl)-4-pyridinyl]amino}-N-methylbenzamide (Intermediate 1, 50 mg, 0.152 mmol) and [2-(methyloxy)-4-(4-morpholinyl)phenyl]amine (148 mg, 0.607 mmol) were combined with 1M hydrochloric acid (0.303 mL, 0.303 mmol), 1,4-dioxane (0.2 mL) and water (2.5 mL) in a microwave tube, then irradiated in a microwave oven at 170° C. for 25 min. The resulting crude material was filtered and the filtrate was purified by reverse-phase HPLC to give the title compound (35.5 mg, 0.071 mmol, 46.7% yield). MS: M(C₂₅H₂₆F₃N₅O₃)=471.48, (M+H)⁺=472; 1H NMR (400 MHz, METHANOL-d4) δ ppm 2.88 (s, 3 H), 3.05-3.14 (m, 4 H), 3.78-3.91 (m, 7 H), 6.45 (s, 1 H), 6.52 (dd, J=8.59, 2.53 Hz, 1 H), 6.64 (d, J=2.53 Hz, 1 H), 7.03-7.13 (m, 1 H), 7.30 (d, J=8.59 Hz, 1 H), 7.40-7.50 (m, 2 H), 7.61 (dd, J=7.83, 1.26 Hz, 1 H), 8.10 (s, 1 H).

### Example 4

N-Methyl-2-[(5-methyl-2-{[4-(4-morpholinyl)phenyl]amino}-4-pyridinyl)amino]benzamide

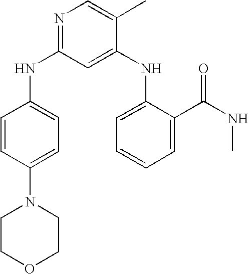

A microwave tube was charged with 4-iodo-5-methyl-N[4-(4-morpholinyl)phenyl]-2-pyridinamine (Intermediate 2, 50 mg, 0.127 mmol), 2-amino-N-methylbenzamide (28.5 mg, 0.190 mmol), palladium(II) acetate (1.420 mg, 6.33 μmol), tripotassium phosphate (67.1 mg, 0.316 mmol) and bis(2-diphenylphosphinophenyl)ether (6.81 mg, 0.013 mmol), then degassed with nitrogen and irradiated in a microwave oven at 180° C. for 20 min. The resulting crude product was purified by reverse-phase HPLC to give the title compound as a TFA salt (42 mg, 59% yield). MS: M(C₂₄H₂₇N₅O₂)=417.50, (M+H)⁺=418; 1H NMR (400 MHz, DMSO-d6) δ ppm 2.17 (s, 3 H) 2.77 (d, J=4.55 Hz, 2 H) 3.04-3.18 (m, 4 H) 3.76-3.74 (m, 4 H) 6.49 (s, 1H) 7.02 (m, J=8.84 Hz, 2 H) 7.14 (m, J=8.84 Hz, 2 H) 7.29 (ddd, J=8.40, 4.11, 3.92 Hz, 1 H) 7.58 (d, J=3.79 Hz, 2 H) 7.61 (s, 1 H) 7.76 (d, J=7.83 Hz, 1 H) 8.73 (m, 1H) 9.57 (s, 1H) 10.24 (s, 1 H).

### Example 5

N-Methyl-2-[(5-methyl-2-{[2-(methyloxy)-4-(4-morpholinyl)phenyl]amino}-4-pyridinyl)amino]benzamide

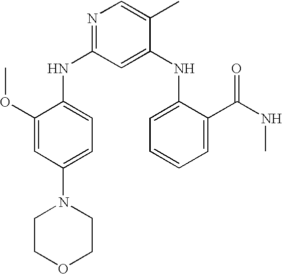

A microwave tube was charged with 4-iodo-5-methyl-N[2-(methyloxy)-4-(4-morpholinyl)phenyl]-2-pyridinamine (Intermediate 3, 50 mg, 0.118 mmol), 2-amino-N-methylbenzamide (19.42 mg, 0.129 mmol), palladium acetate (0.528 mg, 2.351 μmol), tripotassium phosphate (62.4 mg, 0.294 mmol) and bis(2-diphenylphosphinophenyl)ether (5.07 mg, 9.41 μmol). The reaction mixture was degassed with nitrogen for 5 min and irradiated in a microwave oven at 180° C. for 20 min. The resulting crude material was purified by reverse-phase HPLC to give the title compound (36 mg, 59% yield). MS: M(C₂₅H₂₉N₅O₃)=447.53, (M+H)⁺=448; 1H NMR (400 MHz, MeOD) δ ppm 2.25 (s, 3 H) 2.92 (s, 3 H) 3.20-3.22 (m, 4 H) 3.77-3.93 (m, 4 H) 6.52 (s, 1 H) 6.61 (dd, J=8.59, 2.53 Hz, 1 H) 6.70 (d, J=2.53 Hz, 1 H) 7.12 (d, J=8.59 Hz, 1 H) 7.30 (ddd, J=8.02, 6.51, 1.89 Hz, 1 H) 7.44 (s, 1 H) 7.51-7.64 (m, 2 H) 7.72 (d, J=1.01 Hz, 1 H) 8.36 (s, 1 H).

### Example 6

2-[(5-Chloro-2-{[4-(4-morpholinyl)phenyl]amino}-4-pyridinyl)amino]-N-methylbenzamide

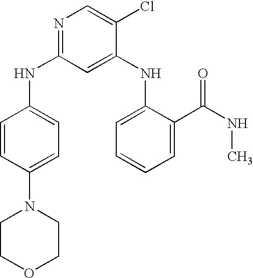

A microwave tube was charged with 5-chloro-4-iodo-N-[4-(4-morpholinyl)phenyl]-2-pyridinamine (Intermediate 4, 55.4 mg, 0.133 mmol), 2-amino-N-methylbenzamide (30 mg, 0.200 mmol), tripotassium phosphate (70.7 mg, 0.33 mmol) and bis(2-diphenylphosphinophenyl)ether (7.17 mg, 0.013 mmol). The reaction mixture was degassed with nitrogen and irradiated in a microwave oven at 180° C. for 20 min. The resulting crude product was purified by reverse-phase HPLC to give the title compound (11 mg, 16% yield). MS: M(C₂₃H₂₄ ClN₅O₂=437.92, (M+H)⁺=438; H NMR (400 MHz, DMSO-d6) δ ppm 2.78 (d, J=4.29 Hz, 3 H) 2.99-3.11 (m, 4 H) 3.72-3.74 (m, 4 H) 6.75 (s, 1 H) 6.86 (m, J=8.84 Hz, 2 H) 7.11 (dd, J=15.16, 1.01 Hz, 1H) 7.41 (m, J=9.09 Hz, 2 H) 7.47-7.63 (m, 2 H) 7.70 (dd, J=7.83, 1.26 Hz, 1 H) 7.99 (s, 1H) 8.64-8.73 (m, 2 H) 10.09 (s, 1 H).

### Example 7

2-[(5-Chloro-2-{[2-(methyloxy)-4-(4-morpholinyl)phenyl]amino}-4-pyridinyl)amino]-N-methylbenzamide

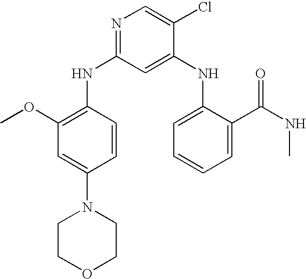

A microwave vessel was charged with 5-chloro-4-iodo-N-[2-(methyloxy)-4-(4-morpholinyl)phenyl]-2-pyridinamine (Intermediate 5, 43 mg, 0.096 mmol), 2-amino-N-methylbenzamide (Intermediate 5, 14.49 mg, 0.096 mmol), and cesium carbonate (94 mg, 0.289 mmol) in 1,4-dioxane (2.5 mL). The reaction mixture was degassed with argon for 2 min, then charged with palladium(II) acetate (2.166 mg, 9.65 μmol) and (±)-2,2′-bis(diphenylphosphino)-1,1′-binaphthalene (12.02 mg, 0.019 mmol), and heated in a microwave oven at 180° C. for 20 min. The mixture was then reduced to dryness in the presence of silica gel (300 mg) and the resulting crude product was added to a silica gel column and eluted with CH₂Cl₂ and chloroform/methanol/ammonium hydroxide (90:9:1) (5% to 100%), to afford the title compound (26 mg, 57% yield). 1H NMR (400 MHz, DMSO-d₆) δ ppm 10.10 (s, 1 H), 8.66 (d, J=4.29 Hz, 1 H), 7.93 (s, 1 H), 7.89 (s, 1 H), 7.68 (d, J=6.82 Hz, 1 H), 7.63 (d, J=8.84 Hz, 1 H), 7.57-7.51 (m, 1 H), 7.48 (t, J=7.07 Hz, 1 H), 7.07 (t, J=7.07 Hz, 1 H), 6.75 (s, 1 H), 6.61 (d, J=2.53 Hz, 1 H), 6.44 (dd, J=8.72, 2.40 Hz, 1 H), 3.80 (s, 3 H), 3.75-3.73 (m, 4 H), 3.07-3.05 (m, 4 H), 2.78 (d, J=4.55 Hz, 3 H); MS: M(C₂₄H₂₆ClN₅O₃)=467.95, (M+H)⁺=468 and 469.

### Example 8

N-Methyl-2-({5-methyl-2-[(2-oxo-2,3-dihydro-1H-indol-5-yl)amino]-4-pyridinyl}amino)benzamide

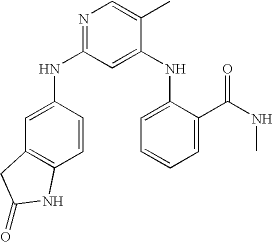

A vessel was charged with 2-[(2-fluoro-5-methyl-4-pyridinyl)amino]-N-methylbenzamide (Intermediate 6, 33 mg, 0.127 mmol) and 5-amino-1,3-dihydro-2H-indol-2-one (37.7 mg, 0.255 mmol) in 1,4-dioxane (0.1 mL) and water (1.5 mL). The reaction mixture was heated at 150° C. for 2 days to produce a crude product, which was purified by reverse-phase HPLC to give the title compound as a solid (208 mg, 48% yield). MS: M(C₂₂H₂₁N₅O₂)=433.46, (M+H)⁺=434; 1H NMR (400 MHz, MeOD) δ ppm 2.25 (s, 3 H) 2.91 (s, 3 H) 3.51-3.58 (m, 1 H) 6.56 (s, 1 H) 6.95 (d, J=8.34 Hz, 1 H) 7.14 (dd, J=8.21, 1.89 Hz, 1 H) 7.22 (d, J=1.52 Hz, 1 H) 7.27 (dd, J=14.91, 1.52 Hz, 1 H) 7.45-7.64 (m, 2 H) 7.71 (dd, J=7.83, 1.26 Hz, 1 H) 8.45 (s, 1 H).

### Example 9

2-[(5-Fluoro-2-{[2-(methyloxy)-4-(4-morpholinyl)phenyl]amino}-4-pyridinyl)amino]-N-methylbenzamide

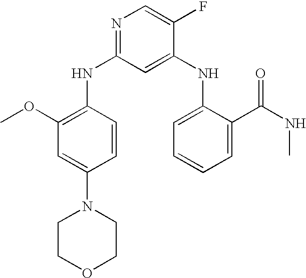

A microwave vessel was charged with 2-[(2-chloro-5-fluoro-4-pyridinyl)amino]-N-methylbenzamide (Intermediate 7, 54 mg, 0.193 mmol), [2-(methyloxy)-4-(4-morpholinyl)phenyl]amine hydrochloride (47.2 mg, 0.193 mmol), and cesium carbonate (189 mg, 0.579 mmol) in 1,4-dioxane (2.5 mL). The reaction mixture was bubbled with argon for 2 min, then charged with palladium(II) acetate (2.167 mg, 9.65 μmol), and (±)-2,2′-Bis(diphenylphosphino)-1,1′-binaphthalene (12.02 mg, 0.019 mmol) and irradiated in a microwave oven at 180° C. for 20 min. The reaction mixture was reduced to dryness in the presence of silica gel (300 mg) and the resulting crude product was added to a silica gel column and eluted with hexanes and EtOAc (5% to 100%) then purified by reverse-phase HPLC to afford the title compound (5 mg, 5% yield). MS: M(C₂₄H₂₆FN₅O₃)=451.49, (M+H)⁺=452 and 453.

### Example 10

2-({5-Chloro-2-[(2-oxo-2,3-dihydro-1H-indol-5-yl)amino]-4-pyridinyl}amino)-N-methylbenzamide

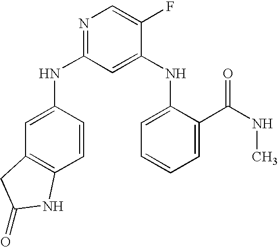

A microwave vessel was charged with 2-[(2-chloro-5-fluoro-4-pyridinyl)amino]-N-methylbenzamide (Intermediate 7, 80 mg, 0.286 mmol), 5-amino-1,3-dihydro-2H-indol-2-one (42.2 mg, 0.286 mmol) in 1,4-dioxane (0.182 mL) and water (2 mL) and irradiated in a microwave oven at 180° C. for 20 min. The reaction mixture was reduced to dryness in the presence of silica gel (300 mg) and the resultant crude product was added to a silica gel column and eluted with hexanes and EtOAc (5% to 100%) then purified by reverse-phase HPLC to afford the title compound (23 mg, 19% yield). MS: M(C₂₁H₁₈ClN₅O₂)=407.85, (M+H)⁺=408 and 409.

### Example 11

2-[(5-Bromo-2-{[4-(4-morpholinyl)phenyl]amino}-4-pyridinyl)amino]-N-methylbenzamide

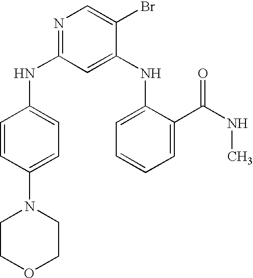

4-(4-Morpholinyl)aniline (47.3 mg, 0.265 mmol) and 2-[(5-bromo-2-chloro-4-pyridinyl)amino]-N-methylbenzamide hydrochloride salt (Intermediate 8, 100 mg, 0.265 mmol) were combined in a vessel with isopropanol (3 mL). 6 M Hydrochloric acid (0.044 mL, 0.265 mmol) was added and the vessel was sealed and heated at 120° C. for 96 h. The reaction mixture was purified by reverse-phase HPLC. The desired fractions were combined and evaporated to give the title compound as a brown oil (15 mg, 10% yield). 1H NMR (400 MHz, MeOD) δ ppm 2.91 (s, 3 H) 3.06-3.13 (m, 4 H) 3.79-3.91 (m, 4 H) 6.62 (s, 1 H) 6.97 (m, J=9.09 Hz, 2 H) 7.10-7.21 (m, 1 H) 7.24 (m, J=8.84 Hz, 2 H) 7.43-7.53 (m, 1 H) 7.53-7.62 (m, 1 H) 7.65 (dd, J=7.71, 1.39 Hz, 1 H) 7.97 (s, 1 H) 8.18 (br. s., 2 H); MS: M(C₂₃H₂₄BrN₅O₂)=481.11, (M+H)⁺=482 and 484.

### Example 12

2-[(5-Bromo-2-{[2-(methyloxy)-4-(4-morpholinyl)phenyl]amino}-4-pyridinyl)amino]-N-methylbenzamide

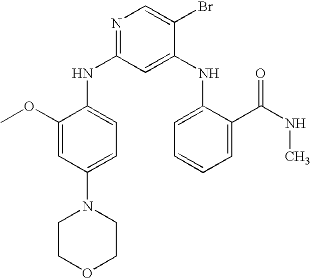

2-(Methyloxy)-4-(4-morpholinyl)aniline (65 mg, 0.265 mmol) and 2-[(5-bromo-2-chloro-4-pyridinyl)amino]-N-methylbenzamide hydrochloride salt (100 mg, 0.265 mmol) (Intermediate 8) were combined in a vessel with isopropanol (3 mL). 6 M Hydrochloric acid (0.044 mL, 0.265 mmol) was added and the reaction mixture was heated at 120° C. for 120 h. The reaction mixture was then purified by reverse-phase HPLC. The desired fractions were combined and evaporated to give the title compound as a purple solid (21 mg, 14% yield). 1H NMR (400 MHz, MeOD) δ ppm 2.88-2.94 (m, 3 H) 3.10-3.19 (m, 4 H) 3.81-3.89 (m, 7 H) 6.51-6.58 (m, 2 H) 6.66 (d, J=2.53 Hz, 1 H) 7.12-7.19 (m, 1 H) 7.29 (d, J=8.59 Hz, 1 H) 7.44-7.51 (m, 1 H) 7.51-7.57 (m, 1 H) 7.64 (dd, J=7.83, 1.26 Hz, 1 H) 7.95 (s, 1 H) 8.29 (br. s., 2 H); MS: M(C₂₄H₂₆BrN₅O₃)=511.12, (M+H)⁺=512 and 514.

### Example 13

2-[(5-Chloro-2-{[2-(methoxy)-4-(4-morpholinyl)phenyl]amino}-4-pyridinyl)amino]-N-(methyloxy)benzamide

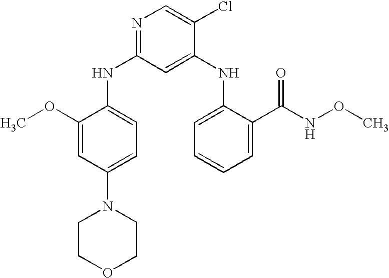

A vessel was charged with 2-[(5-chloro-2-{[2-(methyloxy)-4-(4-morpholinyl)phenyl]amino}-4-pyridinyl)amino]benzoic acid (Intermediate 9, 0.2 g, 0.440 mmol), 1-(3-dimethylaminopropyl)-3-ethylcarbodiimide hydrochloride (0.084 g, 0.440 mmol) and hydroxybenzotriazole (HOBT) (0.067 g, 0.440 mmol) in N,N-dimethylformamide (DMF) (1.0 mL) was stirred at room temperature for 30 min. To this solution methoxylamine hydrochloride (0.037 g, 0.440 mmol) was added and the mixture was stirred for another 10 min before being cooled down to 0° C. by using an ice bath. To this reaction mixture DIEA (0.154 mL, 0.879 mmol) was added and the reaction mixture was stirred at room temperature overnight, The reaction was concentrated under vacuum and the residue was purified by reverse-phase HPLC to give the title compound as a solid (58 mg, 24% yield). MS: M(C₂₄H₂₆ClN₅O₄)=483.95, (M+H)⁺=482, 484; 1H NMR (400 MHz, DMSO-d6) δ ppm 11.89 (br. s., 1 H) 9.64 (br. s., 1 H) 7.93 (s, 1 H) 7.89 (s, 1 H) 7.46-7.67 (m, 4 H) 7.08 (t, J=7.45 Hz, 1 H) 6.67-6.77 (m, 1 H) 6.61 (d, J=2.5 Hz, 1 H) 6.44 (dd, J=8.7, 2.6 Hz,1 H) 3.80 (s, 3 H) 3.72-3.77 (m, 4 H) 3.70 (s, 3 H) 2.98-3.11 (m, 4 H).

The 2-[(5-chloro-2-{[2-(methoxy)-4-(4-morpholinyl)phenyl]amino}-4-pyridinyl)amino]-N-hydroxyalkyl-N-alkylbenzamide compounds illustrated in Table 3 were prepared from 2-[(5-chloro-2-{[2-(methyloxy)-4-(4-morpholinyl)phenyl]amino}-4-pyridinyl)amino]benzoic acid (Intermediate 9) and amino-alcohols substantially according to the procedure of Example 13. The designation *--- represents the point of attachment of the R group.

TABLE 3

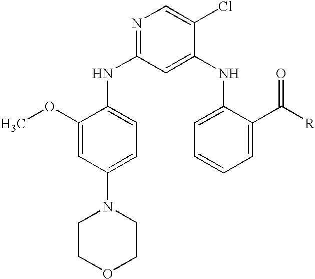

| Ex | Name | R | Data |
|---|---|---|---|
| 14 | 2-[(5-chloro-2-{[2-(methoxy)- 4-(4- morpholinyl)phenyl]amino}-4- pyridinyl)amino]-N-hydroxy- N-methylbenzamide | 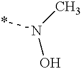 | LC-MS (ES) m/z = 482, 484 (M + H)⁺; 1H NMR (400 MHz, DMSO-d6) δ ppm 8.20 (s, 1H) 7.89 (s, 1H) 7.83 (s, 1H) 7.52-7.61 (m, 2H) 7.40 -7.51 (m, 3H) 7.08-7.20 (m, 1H) 6.59 (d, J = 2.5 Hz, 1H) 6.52 (s, 1H) 6.43 (dd, J = 8.8, 2.5 Hz, 1H) 3.80 (s, 3H) 3.67-3.76 (m, 4H) 3.27 (s, 3H) 2.95-3.11 (m, 4H) |
| 15 | 2-[(5-Chloro-2-{[2- (methyloxy)-4-(4- morpholinyl)phenyl]amino}-4- pyridinyl)amino]-N-methyl-N- (methyloxy)benzamide | 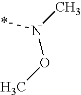 | LC-MS (ES) m/z = 498, 501 (M + H)⁺; 1H NMR (400 MHz, DMSO-d6) δ ppm 7.85-7.92 (m, 2H) 7.76-7.83 (m, 1H) 7.62 (d, J = 8.6 Hz, 1H) 7.46-7.56 (m, 3H) 7.14-7.26 (m, 1H) 6.59 (d, J = 2.5 Hz, 1H) 6.47-6.53 (m, 1H) 6.42 (dd, J = 9.0, 2.7 Hz, 1H) 3.77 (s, 3H) 3.69-3.75 (m, 4H) 3.51 (s, 3H) 3.26 (s, 3H) 3.00-3.09 (m, 4H) |
| 16 | 2-[(5-Chloro-2-{[2-(methoxy)- 4-(4- morpholinyl)phenyl]amino}-4- pyridinyl)amino]-N-{[2- (dimethylamino)ethyl]oxy} benzamide | 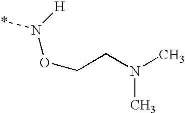 | LC-MS (ES) m/z = 542 (M + H)⁺; 1H NMR (400 MHz, CHLOROFORM-d) δ ppm 9.63 (br. s., 1H) 8.55 (s, 1H) 8.01 (s, 1H) 7.73 (dd, J = 7.8, 1.3 Hz, 1H) 7.46-7.63 (m, 2H) 7.33-7.45 (m, 1H) 7.02-7.11 (m, 1H) 6.88 (br. s., 1H) 6.68 (s, 1H) 6.38-6.59 (m, 2H) 4.27-4.34 (m, 2H) 3.89 (d, J = 4.8 Hz, 2H) 3.86 (s, 3H) 3.06-3.16 (m, 6H) 2.72 (s, 6H) |

### Example 17

2-[5-Cyclopropyl-2-(2-methoxy-4-morpholin-4-yl-phenylamino)-pyridin-4-ylamino]-N-methoxy-benzamide

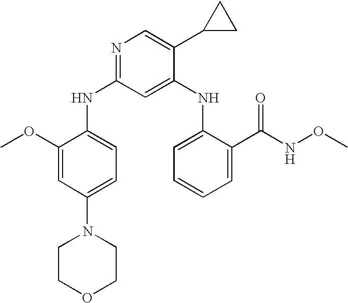

To a 10 mL microwave tube were added 2-(2-chloro-5-cyclopropyl-pyridin-4-ylamino)-N-methoxy-benzamide (Intermediate 12, 0.075 g, 0.24 mmol, 1 eq), 2-methoxy-4-morpholin-4-yl-phenylamine (0.1 g, 0.47 mmol, 2 eq), Cs₂CO₃ (0.23 g, 0.71 mmol, 3 eq), and 1,4-dioxane (3 mL) and resulying mixture was degassed with N₂ for 15 min. To this Pd₂(dba)₃ (0.014 g, 0.014 mmol, 0.06 eq) and Xanthphos (0.03 g, 0.06 mmol, 0.25 eq) were added and again degassed with N₂ for another 30 min. The resulting mixture was irradiated in a microwave at 100° C., 150 W for 30 min. Progress of reaction was monitored by LCMS. After completion of reaction, solvent was removed under reduced pressure and crude compound was purified column chromatography over silica gel (100-200 mesh) using 1% MeOH-DCM as eluant followed by prep HPLC. Solid so obtained was washed with diethyl ether and pentane to give the title compound as gray solid (9 mg, 9%). ¹H-NMR (400 MHz, DMSO-d₆): δ 0.51-0.54 (m, 2H), 0.91-0.95 (m, 2H), 1.50-1.62 (m, 1H), 3.00-3.10 (m, 4H), 3.65-3.72 (m, 3H), 3.72-3.75 (m, 4H), 3.79 (s, 3H), 6.35-6.60 (m, 1H), 6.55-6.62 (m, 1H), 6.67 (s, 1H), 6.95-7.10 (m, 1H), 7.40-7.65 (m, 4H), 7.66-7.76 (m, 2H), 9.47 (s, 1H), 11.85 (brs, 1H). LC-MS calculated for C₂₇H₃₁N₅O₄ [M+H] 490.24, found 490.5. HPLC purity 91.0% at 210 nm.

### Example 18

2-[5-Cyclopropyl-2-(2-methoxy-4-morpholin-4-yl-phenylamino)-pyridin-4-ylamino]-N-methyl-benzamide

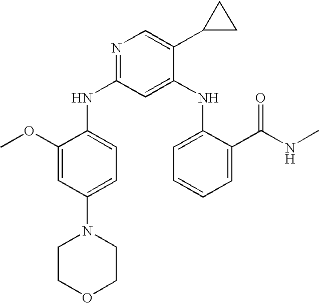

To a 10 mL microwave tube were added 2-(2-chloro-5-cyclopropyl-pyridin-4-ylamino)-N-methyl-benzamide (Intermediate 12, 0.075 g, 0.25 mmol, 1 eq), 2-methoxy-4-morpholin-4-yl-phenylamine (0.10 g, 0.50 mmol, 2 eq), Cs₂CO₃ (0.24 g, 0.75 mmol, 3 eq) and 1,4-dioxane (3 mL) and resulting mixture was degassed with N₂ for 30 minutes. To this Pd₂(dba)₃ (0.010 g, 0.009 mmol, 0.04 eq) and Xanthphos (0.021 g, 0.037 mmol, 0.15 eq) were added and reaction mixture was degassed again with N₂ for another 10 minutes. The resulting reaction mixture was irradiated in CEM microwave at 110° C. and 150 W for 30 min. The progress of reaction was monitored by LCMS. After completion of reaction, Cs₂CO₃ was removed by filtration and filtrate was concentrated under reduced pressure and crude compound was purified by prep HPLC to yield the title compound as a gray solid (7 mg, 6%). ¹H-NMR (400 MHz, DMSO-d₆): δ 0.45-0.60 (m, 2H), 0.90-0.93 (m, 2H), 1.50-1.70 (m, 1H), 2.70-2.80 (m, 3H), 3.95-3.10 (m, 4H), 3.70-3.75 (m, 4H), 3.79 (s, 3H), 6.41-6.42 (d, 1H, J=7.48 Hz), 6.59 (s, 1H), 6.69 (s, 1H), 6.90-7.10 (m, 1H) 7.35-7.46 (m, 1H), 7.50-7.60 (m, 2H), 7.62-7.63 (d, 1H, J=7.4 Hz), 7.68 (s, 1H), 7.71-7.73 (d, 1H, J=6.8 Hz), 8.50-8.65 (brs, 1H), 9.97 (s, 1H). LC-MS calculated for C₂₇H₃₁N₅O₃ [M+H] 474.24, found 474.6. HPLC purity 99.89% at 210 nm.

### Example 19

2-[2-(2-Methoxy-4-morpholin-4-yl-phenylamino)-5-vinyl-pyridin-4-ylamino]-N-methyl-benzamide

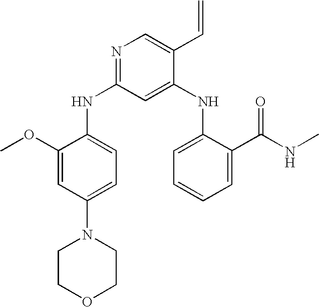

To a sealed tube 2-(2-chloro-5-vinyl-pyridin-4-ylamino)-N-methyl-benzamide (Intermediate 14, 0.5 g, 1.74 mmol, 1 eq), 2-methoxy-4-morpholin-4-yl-phenylamine (0.72 g, 3.47 mmol, 2 eq), Cs₂CO₃ (1.69 g, 5.21 mmol, 3 eq) and 1,4-dioxane (50 mL) were added and the resulting mixture was degassed with N₂ at 50° C. for 1 h. To this Pd(OAc)₂ (0.078 g, 0.35 mmol, 0.2 eq), BINAP (0.43 g, 0.7 mmol, 0.4 eq) were added and again degassed with N₂ for another 30 minutes and resulting mixture was heated at 100° C. for overnight. Progress of reaction was monitored by LCMS. After completion of reaction, Cs₂CO₃ was filtered off, and filtrate was evaporated under reduced pressure to yield the crude product which was purified by column chromatography over silica gel (100-200 mesh) using 0.2% MeOH-DCM as eluant followed by prep HPLC. The solid compound so obtained was washed with diethyl ether and pentane to give the title compound as a brown solid (13 mg, 2%). ¹H-NMR (400 MHz, DMSO-d₆): δ 2.74-2.75 (d, 3H, J=4.36 Hz), 3.00-3.10 (m, 4H), 3.69-3.78 (m, 4H), 3.78 (s, 3H), 5.18-5.25 (m, 1H), 5.60-5.68 (m, 1H), 6.42-6.49 (m, 1H), 6.58-6.73 (m, 3H), 6.95-7.05 (m, 1H), 7.40-7.50 (m, 2H), 7.62-7.70 (m, 2H) 7.81 (s, 1H), 8.06 (s, 1H), 8.60-8.70 (brs, 1H), 9.98 (s, 1H). LC-MS calculated for C₂₆H₂₉N₅O₃ [M+H] 460.23, found 460.4. HPLC purity 98.20% at 210 nm.

### Example 20

2-[(5-Cyano-2-{[2-(methyloxy)-4-(4-morpholinyl)phenyl]amino}-4-pyridinyl)amino]-N-methylbenzamide

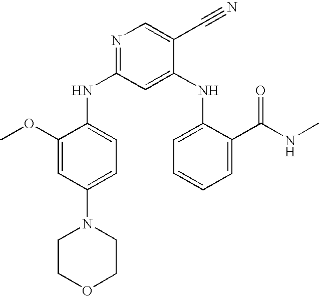

Example 20 was synthesized in a similar fashion as Example 19 but using Intermediate 16 and 2-methoxy-4-morpholin-4-yl-phenylamine. LC-MS calculated for C₂₅H₂₆N₆O₃ (M+H) 459.52, found 459.4. HPLC purity 97.4% at 200 nm.

1. A compound represented by the following formula: 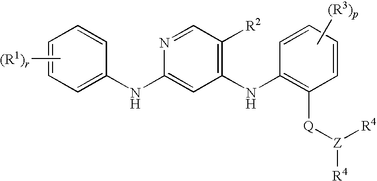
or a pharmaceutically acceptable salt thereof, wherein:
each R¹ is independently halo, hydroxy, cyano, nitro, —COOH, —COO—C₁-C₃-alkyl, C₁-C₆-alkoxy, —(NR⁵)ₓSO_{y}R⁶, —X—N(R⁷)₂, —SO_{y}N(R⁷)₂, —C₁-C₆-alkyl-(R⁸)ₚ, C₃-C₆-cycloalkyl-R⁹, phenyl-(R¹⁰)ₚ, heteroaryl-(R¹⁰)ₚ, heterocycloalkyl-(R¹¹)ₚ, or two ortho R¹ groups, together with the carbon atoms to which they are attached, form a fused 5-6-membered carbocyclic or heterocyclic ring;
R² is halo, CF₃, C₁-C₆-alkyl, C₃-C₆-cycloalkyl, C₁-C₆-alkoxy, or cyano;
each R³ is independently halo, C₁-C₆-alkyl, —C₁-C₆-alkyl-R⁸, C₁-C₆-alkoxy, —X—N(R⁷)₂, or heterocycloalkyl;
each R⁴ is independently H, C₃-C₆-cycloalkyl, —C₁-C₆-alkyl—(R⁸)ₚ, hydroxy, or O—C₁-C₆-alkyl—(R⁸)ₚ, or the R⁴ groups, together with Z, form a 5-6-membered cyclic ring optionally substituted with a —C₁-C₆-alkyl—(R⁸)ₚ group or a C₃-C₆-cycloalkyl group;
each R⁵ is independently H or —C₁-C₆-alkyl—(R⁸)ₚ;
each R⁶ is independently hydroxy, C₁-C₆-alkyl, phenyl—(R¹⁰)ₚ, or heteroaryl—(R¹⁰)ₚ;
each R⁷ is independently H, —(Y)ₓ-C₁-C₆-alkyl—R⁸, C₃-C₆-cycloalkyl, phenyl—(R¹⁰)ₚ, heteroaryl—(R¹⁰)ₚ, heterocycloalkyl—(R¹¹)ₚ, or, together with the nitrogen atom to which they are attached, form a 5-6-membered cyclic ring optionally substituted with a —C₁-C₆-alkyl—(R⁸)ₚ or C₃-C₆-cycloalkyl group;
each R⁸ is independently C₁-C₆-alkoxy, C₃-C₆-cycloalkyl, C₁-C₆-thioalkoxy, hydroxy, thiol, cyano, halo, nitro, —COOH, —COO-C₁-C₃-alkyl, —(NR⁵)ₓSO_{y}R⁶, —X—N(R⁷)₂, —SO_{y}N(R⁷)₂, phenyl-(R¹⁰)ₚ, or heteroaryl-(R¹⁰)ₚ;
each R⁹ is independently C₁-C₆-alkoxy, —COOH, —COO-C₁-C₃-alkyl, —X—N(R⁵)₂, halo, hydroxy, or —C₁-C₆-alkyl-(R⁸)ₚ;
each R¹⁰ is independently C₁-C₆-alkyl, C₁-C₆-alkoxy, hydroxy, halo, CF₃, or N(R⁵)₂;
each R¹¹ is independently C₁-C₆-alkyl-(R⁸)ₚ;
Q is —C(O)—, —S(O)—, or —SO₂—;
Z is N, CH, or C-C₁-C₆-alkyl;
X is a bond, —C₁-C₆-alkyl—, —C(O)—, or —O—(CH₂)_{q}—;
Y is —S(O)—, —SO₂—, —C(O)—, —C(O)NH—, or —C(O)O—;
each p is independently 0, 1, 2, or 3;
q is 1, 2, 3, or 4;
r is 0, 1, 2, or 3;
each x is independently 0 or 1; and
each y is independently 1 or 2.

2. The compound of claim 1 which is represented by the following structure: 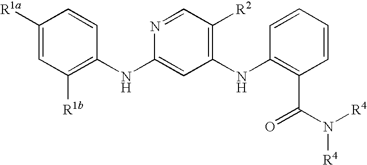
or a pharmaceutically acceptable salt thereof, wherein
R^{1a} is heterocycloalkyl-(R¹¹)ₚ, —C₁-C₆-alkyl-R⁸, or C₁-C₆-alkoxy;
R^{1b} is H, C₁-C₃ alkoxy, halo, CF₃, SO_{y}C₁-C₃-alkyl, or —C(O)N(R⁷)₂;
R² is halo or CF₃;
each R⁴ is independently H methyl, or methoxy; and
each R⁷ is independently H, C₁-C₃-alkyl, or, together with the nitrogen atom to which they are attached form a pyrrolidinyl, piperidinyl, piperazinyl, morpholino, or thiomorpholino group.

3. The compound of claim 1 which is represented by the following structure: 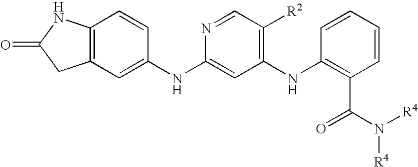
or a pharmaceutically acceptable salt thereof, wherein
R² is halo or CF₃; and
each R⁴ is independently H or methyl.

4. The compound of claim 1 which is represented by the following structure: 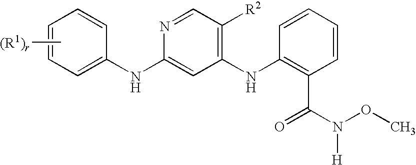
or a pharmaceutically acceptable salt thereof.

5. The compound of claim 2, or a pharmaceutically acceptable salt thereof, wherein one R⁴ is H and the other R⁴ is methoxy.

6. The compound of claim 2, or a pharmaceutically acceptable salt thereof, wherein R^{1a} is a morpholino group and R^{1b} is a methoxy group.

7. The compound of claim 1, or a pharmaceutically acceptable salt thereof, wherein R² is halo or CF₃.

8. A composition comprising a) the compound of claim 1 or a pharmaceutically acceptable salt thereof; and b) a pharmaceutically acceptable excipient.

9. A method of treating cancer comprising administering to a patient in need thereof a pharmaceutically effective amount of the compound of claim 1.

10. A method of treating cancer comprising administering to a patient in need thereof a pharmaceutically effective amount of the composition of claim 8.
